(19) United States
(12) Patent Application Publication
(10) Pub. No.: US 2009/0328239 A1
(43) Pub. Date: Dec. 31, 2009

(54) BLOOD VESSEL IMAGING AND USES THEREFOR

(75) Inventors: Raul A. Brauner, Framingham, MA (US); John Heymach, Pearland, TX (US); George Naumov, Bookline, MA (US); Kongbin Kang, Providence, RI (US)

Correspondence Address: WOLF GREENFIELD & SACKS, P.C., 600 ATLANTIC AVENUE, BOSTON, MA, 02210-2206, US

(73) Assignee: Bio Tree Systems, Inc., Framingham, MA (US)

(21) Appl. No.: 12/363,283

(22) Filed: Jan. 30, 2009

Related U.S. Application Data
(63) Continuation of application No. PCT/US2007/017197, filed on Jul. 30, 2007.
(60) Provisional application No. 60/834,988, filed on Jul. 31, 2006.

(51) Int. Cl. A61K 49/00; G01N 33/00; C12Q 1/00; A61P 43/00

(52) U.S. Cl. 800/3; 424/9.1; 435/4

(57) ABSTRACT

Methods are disclosed for analyzing representations of one or more structures in the body of a subject (e.g., a human subject or other animal subject) to glean information about the health of the subject or to evaluate the subject's response to a therapy or other condition. Aspects of the invention relate to obtaining structural information from casts (e.g., vascular casts from animal models) and using the information as a reference for evaluating structures in the body of a subject. Methods are disclosed for diagnosing, staging, grading, and monitoring diseases. Methods also are disclosed for targeting treatments, monitoring the effectiveness of therapies, and/or screening or validating therapies based analyzing structures (e.g., vascular structures) in a subject and comparing them to reference structures observed in casts obtained from models (e.g., animal models) of related diseases or conditions.

### RELATED APPLICATIONS

This application is a continuation of PCT/US2007/017197, filed Jul. 30, 2007, which claims benefit under 35 U.S.C. 119(e) of the filing date of U.S. provisional application Ser. No. 60/834,988, filed Jul. 31, 2006, the contents of each of which are incorporated by reference herein in their entirety.

### FIELD OF THE INVENTION

Aspects of the present invention relate to analyzing images for diagnostic and therapeutic applications in animals. In particular, aspects of the invention relate to analyzing images to identify structural features in animal bodies for detecting, monitoring, and/or treating diseases, and/or for evaluating and validating new therapies.

### BACKGROUND OF THE INVENTION

A wide range of imaging methods and devices are commonly used to evaluate different anatomical and physiological conditions in a variety of medical and research environments. Tools have been developed to image body structures based on different physical properties. For example, X-rays, CT scans, MRIs, PET scans, IR analyses and other technologies have been developed to obtain images of various body structures. These tools are routinely used for diagnostic, therapeutic, and research applications. Combinations of two or more different imaging techniques are sometimes used to provide complementary information about a patient.

### SUMMARY OF THE INVENTION

Aspects of the invention relate to analyzing tubular body structures. The invention provides methods for analyzing structures such as blood vessels and evaluating their association with disease, responsiveness to therapeutic treatments, and other conditions. In some aspects, vascular casts are analyzed to identify one or more blood vessel structural features (including, for example, abnormal excess or absence of blood vessels or blood vessel structures) that are associated with a disease or other condition of interest. Structural features identified in casts may be used as biomarkers or references to evaluate in situ vasculature, for example, to detect indicia of a disease or other condition of interest in a subject. Structural characteristics of vascular casts also may be used to evaluate therapeutic treatments, screen candidate compounds, and for other applications as described in more detail herein. In some embodiments, one or more structural parameters are analyzed over time (e.g., using a series of vascular casts obtained at different time points) to monitor and/or identify structural changes that occur during development, disease progression or regression, or in response to therapy. In some embodiments, structural analysis is performed on vascular casts obtained from experimental models (e.g., whole animal models, or organ or tissue models). However, in some embodiments, vascular casts are obtained and analyzed for one or more regions of interest (e.g., diseased regions) in dead animals, including for example dead humans (e.g., human cadavers).

In some embodiments, structural parameters and/or structural changes observed for vascular casts from experimental animals (or organs or tissues) can be used as references when analyzing vasculature in vivo. For example, structural vasculature parameters and/or changes that are identified in casts using experimental animal models subsequently can be detected or monitored in vivo (e.g., in a human subject) and used to evaluate the development of a disease, a drug response or other biological or disease property associated with the vasculature parameters and/or changes in a subject. In some embodiments, structural characteristics identified in vascular casts may be used to identify one or more patient subpopulations that are (or are predicted to be) more responsive to a particular treatment. For example, responsive subjects may be identified as those having one or more blood vessel characteristics that were associated with responsiveness in animal models and identified by analyzing vascular casts from the responsive animals. For example, one or more of the following non-limiting structural characteristics (e.g., combinations of 2, 3, 4, 5, 6, 7, 8, 9, 10 or all of the following structural characteristics) may be evaluated (e.g., quantified) in vascular casts: mean vessel diameter distribution, vessel branching distribution, angle of vessel branching distribution, interbranching distances, vessel density, vessel tortuosity, intervessel distances, luminal vessel surface, vessel dilation (changes in vessel diameter over a segment), sinosoidalation (dilation in sinosoids), or permeabilization (vessel leakiness). However, it should be appreciated that other structural characteristics, for example, other characteristics described herein also may be analyzed in vascular casts. One or more of these characteristics, or combinations of characteristics, or related structural changes over time, may be identified as structural patterns that can be associated with one or more conditions of interest. Once identified, these patterns can be used as biomarkers to identify or monitor the conditions of interest in vivo in a subject, for example, by analyzing the in situ vasculature of the subject (or a portion thereof) and detecting the presence of and/or quantifying the extent of a specific vascular structural pattern.

Structural biomarkers of the invention can be quantified and compared using standard statistical methods. These biomarkers can be compared on individual basis, but also in combination as a signature of vascular morphology and function. Whole signatures can be compared between treated and untreated samples, or samples with physiological and pathological vascular pattern.

Aspects of the invention may include the analysis of one or more regions of interest in animal disease models (e.g., in situ and/or in casts of one or more regions of interest). Animal disease models may be, but are not limited to, engineered (e.g., recombinant) animals, transgenic animals, metastatic cancer models, xenograft models, orthotopic transplant models, etc., or any combination thereof. Any suitable animal may be used as an animal model, including, but not limited to, a mouse, rat, hamster, guinea pig, pig, dog, cat, rabbit, zebrafish, or other suitable animal. It should be appreciated that whole experimental animals may be analyzed. However, in some embodiments, tissues and/or organs may be analyzed. In some embodiments, models may be based on xenografts (e.g., xenografts of cancer or tumor cells that will form cancer or tumor tissues in a host animal). For example, human cells may be introduced into a non-human host animal. Other uses of xenografts include analyzing responses to certain tissue and/or organ transplantation (e.g., a non-human tissue or organ into a human host). In some embodiments, vascular casts of regions of interest in an animal model may be obtained to thoroughly analyze the vascular structures, and/or changes therein, associated with the condition being modeled. In some embodiments, observations made on casts may be compared (e.g., using appropriate statistical techniques) to in vivo (e.g., in situ) observations to identify one or more common structural characteristics and/or changes that are statistically significant in vivo in association with a disease, condition, or response of interest. These can then be used in subsequent applications as described herein.

As used herein, a vascular cast refers to a physical structure that is generated to represent blood vessels of an entire vasculature or portion thereof. A cast may be obtained by perfusing a vasculature or a vascular region (e.g., the blood vessels of an organ, for example, of a kidney or liver) with a casting material that solidifies (e.g., polymerizes) to form a stable structure. The surrounding tissue and cells (e.g., including the blood vessel walls) may be removed to reveal the cast. The cast retains the structural features of the original blood vessels. Cast may include structures of blood vessels of different sizes as described herein. Certain casts are more flexible than others, certain casts are more brittle than others. Vascular casts can be used to identify vascular structural features with high resolution and/or to identify correlations between structural features and conditions of interest with high degrees of confidence since the structures of the blood vessels are retained in the casts and other biological structures that could interfere with an analysis are removed. Vascular casts may be obtained using any suitable casting material. In some embodiments, the casting agent may be a polymer. In some embodiments, the casting agent may react with the blood vessel walls. Non-limiting examples of casting agents include, but are not limited to Microfil®, methyl methacrylate, prepolymerized methyl methacrylate (Mercox™), Mercox™ CL-2B, other acrylic resins, silicon, gold nanoparticles, Batson No. 17, polyurethane-based casting agents (e.g., PU4ii), etc., or combinations of two or more thereof.

It should be appreciated that casting agents may be supplemented with contrast agents and/or other detectable agents. Examples of contrast agents include, but are not limited to, BaSo₄ and UAc (e.g., mixed into the casting material). In some embodiments, already polymerized casts can be soaked in OSO₄ to achieve better contrast using CT imaging. In certain embodiments, any suitable heavy metal can be mixed into the resin to make it more radioopaque.

In some embodiments, data for tubular structures (e.g., blood vessels) may been sorted into bins based on their size (e.g., their diameter). Aspects of the invention may increase the analytical resolution when evaluating structural information that is obtained for one or more experimental models and/or subjects being evaluated. According to aspects of the invention, a binned structural analysis refers to any analysis of tubular structures that have been sorted or categorized according to size (e.g., according to the diameter or radius of the tubular structure in an area of interest). For example, in some embodiments a binned micro-vessel density (BMVD) analysis refers to an analysis of blood vessel density based on blood vessels that have been categorized according to vessel diameter in an area of interest.

Binned analytical techniques can be applied to the analysis of many different parameters that may be characteristic of tubular structures. Binned analytical techniques may be performed on tubular structures observed in casts or in vivo (e.g., in situ). For example, bins of tubular structures having different diameters can be evaluated to determine one or more of the following parameters: tortuosity, curvature, density, branching frequency, branching hierarchy (e.g., presence or absence of a branching hierarchy), relative distribution and/or direction of tubular structures (e.g., blood vessels), etc., or any combination thereof. By performing the analysis on binned data, small changes that primarily affect structures in one size range are more likely to be detected, because they are not masked by a relative absence of change in structures in other size ranges. Accordingly, methods of the invention can be used to refine an analysis of tubular structures (e.g., blood vessels) over time or in response to disease or treatment, etc., where the analysis may be performed on casts and/or in vivo. Aspects of the invention can also be used to detect or delineate diseased tissue (e.g., cancerous or pre-cancerous tissue, necrotic regions, etc.) in casts and/or in vivo.

Accordingly, aspects of the invention provide methods and devices for obtaining and/or analyzing data relating to internal tubular structures in casts and/or in human and/or other animal bodies. In some embodiments, methods of the invention involve analyzing one or more parameters (or parameter changes over time) for binned blood vessels that have been categorized based on their size. For example, blood vessels may be binned according to the following non-limiting diameter ranges: about 0-10 microns, about 10-25 microns, about 25-50 microns, about 50-75 microns, about 75-100 microns, about 100-150 microns, about 150-200 microns, about 200-300 microns, about 300-400 microns, about 400-500 microns, about 500-1,000 microns, or any combination thereof. However, any other suitable bin size ranges (including larger, smaller, or intermediate size ranges) may be used. In some embodiments, the number of different bins may be between about 2 and about 10. However, higher numbers of bins also may be used. In some embodiments, only 2 to 5 bins are used (e.g., 2, 3, 4, or 5). In certain embodiments, three blood vessel bin sizes are used: small, medium, and large. In some embodiments, a single bin is chosen having a predetermined size range and no other size ranges are analyzed.

Data relating to one or more selected structures (e.g., structural patterns obtained from an analysis of a vascular cast) may be obtained and/or analyzed to glean information about a physiological condition of an animal based on the structure (or changes in the structure). For example, patterns identified in casts may be used as biomarkers to screen in situ vasculatures for the presence of one or more similar patterns or to quantify the extent of the pattern in situ. This information may be used for diagnostic, predictive, prognostic, therapeutic, interventional, research and/or development purposes, as well as for grading and/or staging a disease. In some embodiments, methods of the invention may involve analyzing one or more structural parameters (or one or more structural parameter changes over time) based on binned structure data or information obtained for casts (e.g., vascular casts) or in situ structures (e.g., in vivo blood vessels).

In some embodiments, one or more structures and/or structural changes that are identified using casts may be detected or monitored in vivo to determine whether a predetermined disease, condition, or response is present in vivo.

Tubular structures (e.g., blood vessels in a cast or in vivo) of different size ranges may be analyzed separately and compared to different threshold or reference values as described herein. In some embodiments, one or more structural parameters are obtained (e.g., calculated or modeled, etc.) for only a subset of size ranges (e.g., only for those size ranges for which changes are known to be associated with a diagnostic, prognostic, clinical, or research application of interest). However, in certain embodiments, all of the size ranges are analyzed. In some embodiments, one or more different parameters are analyzed for different size ranges. However, in certain embodiments, the same parameter(s) is/are analyzed for all of the size ranges that are being assayed. Analyses may be provided in the form of histograms or curves representing a distribution of numerical values or scores obtained for the different ranges.

Certain aspects of the invention are described in more detail in the following sections including the Examples and Figures. It should be appreciated that analytical techniques used to categorize blood vessels based on size may be used to categorize other tubular body structures based on size. In some embodiments, once the tubular structures (e.g., blood vessels) are categorized based on size, the associated values or scores obtained for different parameters of interest can also be categorized and analyzed.

Aspects of the invention may be automated, for example, as described herein.

### BRIEF DESCRIPTION OF THE DRAWINGS

FIG. 1 illustrates blood vessel size distribution in an example of casts of a xenograft tumor model after treatment with Avastin® (an anti-angiogenic agent available from Genentech, South San Francisco, Calif.), in accordance with some embodiments of the present invention;

FIG. 2 illustrates the vessel population ratio between small and middle size vessels in an example of casts of a xenograft tumor model after treatment with Avastin®, in accordance with some embodiments of the present invention; and,

FIG. 3 illustrates the vessel population ratio between large and middle size vessels in an example of casts of a xenograft tumor model after treatment with Avastin®, in accordance with some embodiments of the present invention.

### DESCRIPTION OF THE INVENTION

Aspects of the invention relate to analyzing data obtained for body structures in animals (e.g., in test animals). In one embodiment, the invention relates to obtaining pattern information relating to one or more aspects or regions of the vasculature of an animal. Pattern information obtained according to aspects of the invention may be used to analyze a disease model (e.g., to assess whether an animal disease model is representative of an actual disease based on structural vascular features, or to assess the progression of one or more vascular changes in a test animal that provides a validated disease model, etc.), to evaluate the effectiveness of a treatment regimen, to identify candidate compounds or treatment regimens that are therapeutically effective, or for other applications where data relating to vascular structures (e.g., the progression of vascular structures, changes in vascular structure over time or in response to different drugs or drug dosages or administration frequencies, etc., or any combination thereof) is informative. For example, aspects of the invention may be used to identify one or more pattern elements that can be used to diagnose or evaluate diseases, monitor treatments, screen therapeutic agents, etc., or any combination thereof.

In one aspect, a tissue or organ of an animal is perfused with a composition comprising a casting agent to stabilize one or more vascular structures in the tissue or organ. Subsequently, the vascular structure(s) are analyzed (e.g., by analyzing the structure of the cast). In some embodiments, a casting agent may also be a contrast agent for the technique that is used to image or otherwise detect the vascular structure. In certain embodiments, the casting agent is not a contrast agent. In some embodiments, the tissue or organ may be perfused with an additional contrast agent may (e.g., a contrast agent may be added to the composition comprising the casting agent).

In some embodiments, the casting agent may be a polymer. In some embodiments, the casting agent may react with the blood vessel walls. Non-limiting examples of casting agents include, but are not limited to Microfil®, methyl methacrylate, prepolymerized methyl methacrylate (Mercox™), Mercox™ CL-2B, silicon, gold nanoparticles, Batson No. 17, etc., or combinations of two or more thereof. In some embodiments, a large volume of an animal body (e.g., the entire body) may be perfused with a casting agent composition. In certain embodiments, a small volume of an animal (e.g., a tissue, an organ or a region of either one thereof) may be perfused with a casting agent composition. In some embodiments, a casting agent may be perfused into a tissue or an organ or a region of either one thereof after removal from an animal (e.g., after biopsy or other surgical excision). In some embodiments, a casting agent composition may be perfused into a live animal. It should be appreciated that an animal may be sacrificed after perfusion with a casting agent depending, in part, on the amount and type of casting agent composition that is used and the tissue or organ to which the casting agent composition is targeted. According to aspects of the invention, casting agent(s) may be used to preserve in vivo structures for detailed analysis. In some embodiments, this analysis identifies particular structural or distribution properties that can be subsequently used as markers for in vivo diagnostic, therapeutic, research, and/or other applications in live animals (including humans).

In some aspects, vascular structures may be analyzed in situ in an animal after perfusion with a casting agent composition. In some aspects, a tissue or an organ or a region of either one thereof may be removed from an animal for analysis (e.g., before or after perfusion with a casting agent composition).

Accordingly, aspects of the invention can be used to represent and/or visualize blood vessels with a casting agent or medium.

Aspects of the invention may be used to study, identify, and or analyze structural features of blood vessels that are associated with one or more diseases or conditions represented by an animal of interest. In some embodiments, an animal may be a disease model as described herein. In some embodiments, an animal may be undergoing a therapeutic regimen of interest. In some embodiments, an animal may be treated with a candidate therapeutic compound. Accordingly, aspects of the invention may be used to identify, analyze, and/or evaluate one or more vascular patterns or changes in vascular patterns associated with a disease. Aspects of the invention also may be used to evaluate the effects of one or more therapeutic regimens or candidate compounds. In some embodiments, therapeutic effectiveness may be evaluated using one or more vascular patterns or changes therein as a marker of a response (or lack thereof) to treatment. Accordingly, aspects of the invention may be used to identify particular vascular patterns that are indicative of certain diseases or disease stages. These patterns can subsequently be used in sensitive assays to detect diseases in vivo (e.g., in human subjects). Other aspects of the invention may be used to select therapeutic regimens or candidate compounds for administration to a patient (e.g., a human patient) in a therapeutically effective amount and in a physiologically acceptable form.

It should be appreciated that in some embodiments, an animal that is perfused with a casting agent composition may be sacrificed prior to analysis regardless of whether the analysis is performed in situ or not. Accordingly, in some embodiments, changes over time may be studied using a plurality of animals and using one or more animals for each time point of interest. In some embodiments, different dosages, different therapeutic regimens, different drugs or drug combinations, or any combination of two or more thereof may be studied using different animals (with at least one animal for each condition of interest). It should be appreciated that combinations of time courses and drugs, drugs dosages, or other therapeutic regimens similarly may be studied using a plurality of different animals, each representing a unique condition. It should be appreciated that the different animals are preferably genetically identical or similar (e.g., identical for at least one trait that is associated with a disease or condition of interest). In some embodiments, the animals may be mice, rats, sheep, cats, dogs, primates, or any suitable non-human experimental animal.

In some embodiments, a combination of different drugs, different doses, etc., may be evaluated at a series of time points according to aspects of the invention. Again, it should be appreciated that a different animal may represent a different drug, dosage, time point, or combination thereof, because each animal may be sacrificed for analysis. However, in some embodiments, a single animal may be tested at different sites (representing, e.g., different drugs, dosages, time points, etc.) depending on the impact of the casting agent that is used and the site of administration of the casting agent.

In some embodiments, samples from one or more animals may be prepared and analyzed periodically during the time course of a treatment (e.g., using a group of animals exposed to the same experimental conditions). In some embodiments, different conditions may be compared. For example, separate groups of animals (e.g., groups of mice) may be exposed to a candidate drug and a placebo (or other control). Subsets of animals (e.g., one or more animals) may be perfused with a casting agent composition at different time points and vascular structures may be imaged (e.g., directly or through reconstruction) for each time point. For example, tumors may be induced in genetically-altered mice using appropriate controls and different dose levels or regimens (e.g., 1, 2, 3, 4, 5, or more different dose levels or regimens) of one or more therapeutic compounds or compositions. Vascular structures then may be analyzed at different time points using methods of the invention to evaluate the effectiveness of a drug composition and/or to identify biological markers that can be used to monitor a patient response to the drug composition. It should be appreciated that vascular structures of different sizes may be studied to identify structural features and/or distribution patterns of interest. In some embodiments, blood vessels having a diameter of about 50 microns are studied. However, it should be appreciated that smaller or larger vessels, or a combination thereof, may be studied.

In some embodiments, a structural characteristic may be evaluated over time by comparing results at different time points. However, it should be appreciated that the end-point of a study may be used as a single time point and structural characteristics associated with different diseases or treatments may be compared to identify or infer changes associated with a disease, treatment, or other condition of interest. Aspects of the invention can be used to analyze data obtained from any suitable image source to identify one or more patterns associated with tubular structures of different sizes (e.g., structural patterns of blood micro-vessels). One or more parameters of a structural pattern can be used as biomarkers for different biological conditions and processes (including pathogenic conditions). Accordingly, aspects of the invention relate to disease detection, diagnosis, grading, staging, disease monitoring, monitoring the effectiveness of therapy and interventional applications based on an analysis of structures (e.g., in situ structures) to identify patterns that may be associated or correlated with a disease or other physiological condition. According to the invention, a pattern may comprise one or more different parameters. Parameters may be one or more structural features of individual tubular structures and/or one or more distribution properties (e.g., spatial distribution, spatial orientation, frequency, number, etc., or any combination thereof) of one or more tubular structures and/or one or more distribution properties (e.g., spatial distribution, spatial orientation, frequency, number, etc., or any combination thereof) of one or more individual tubular structural features within a subject or a within a region of interest in the subject, or any combination thereof. Accordingly, a vasculature pattern may include one or more structural features of an individual blood vessel (e.g., micro-vessels), a distribution of one or more blood vessels (e.g., micro-vessels) within a subject, a distribution of one or more individual blood vessel structural features (e.g., individual micro-vessel structural features), or any combination thereof. An individual blood vessel structural feature may include, but is not limited to, vessel tortuosity, curvature, branching (e.g., frequency, angle, hierarchy, etc.), diameter, direction, etc., or any change (e.g., variation or frequency) of any of these features over a predetermined length of the blood vessel being analyzed, or any combination thereof. A distribution of blood vessels or individual blood vessel structural features may include, but is not limited to, a blood vessel density, a distribution of blood vessel directions, a distribution of blood vessel diameters, a distribution of distances between blood vessels, a distribution of blood vessel spatial orientations (e.g., relative to each other), a distribution of blood vessel curvatures, a distribution of any other individual blood vessel structural features described herein, other distributions of blood vessel parameters or any combination of two or more thereof. It should be appreciated that the distribution of blood vessels or blood vessel structural features may be determined and/or analyzed for a predetermined region within a subject (e.g., a target volume of tissue within a subject) or within predetermined tissues or organs within a subject or throughout the subject (e.g., within a vascular cast). It also should be appreciated that either the absence or presence of blood vessels or of individual blood vessel structural features within a predetermined volume being analyzed may be a pattern parameter that can be used in analytical methods of the invention. It also should be appreciated that one or more pattern parameters may be monitored and/or analyzed as a function of time. Accordingly, blood vessel patterns can be used as biomarkers for different biological conditions and processes (including pathogenic conditions). Accordingly, aspects of the invention relate to identifying and evaluating biological markers that may be used for in vivo disease detection, diagnosis, grading, staging, for disease monitoring, for monitoring the effectiveness of therapy and interventional applications in live animals, including humans, based on an analysis of vasculature patterns including vasculature morphology and/or architecture in experimental animals perfused with one or more casting agent compositions. In one embodiment, the in vivo density, and/or diameter distribution, and/or geometric orientation of blood vessels (e.g., micro-vessels) may be analyzed, quantified, and/or evaluated for disease detection, monitoring, and/or interventional applications. In one embodiment, the sensitivity and specificity of disease diagnosis may be enhanced by analyzing and evaluating in vivo vasculature morphology and/or architecture associated with a tissue lesion. Accordingly, aspects of the invention include detecting in vivo indicia of diseases associated with abnormal vascular structures or patterns. Other aspects include disease diagnosis, staging, grading, monitoring and prognosis, patient treatment, drug development and validation, and research applications. It should be appreciated that one or more biological markers identified in vascular casts in association with a response to a known drug or treatment may be used as a reference markers to evaluate the effectiveness of additional drugs or treatments in comparison to the known drug or treatment.

One embodiment according to the present invention includes a method of analyzing geometric features of blood vessels and correlating one or more features with a biological process, condition, or disease. Accordingly, certain geometric features of blood vessels may be used as biomarkers indicative of particular biological processes, conditions, and/or diseases.

Aspects of the invention relate to business methods that may involve the marketing and/or licensing of biomarkers associated with particular biological processes, conditions, and/or diseases. In some embodiments, patterns (e.g., geometric features) of blood vessels (e.g., observed in casts) are analyzed to identify or evaluate associations or correlations with certain biological processes, conditions, and/or diseases of interest. Pattern parameters may be identified that can be used as structural biomarkers (e.g., for clinical, diagnostic, therapeutic, and/or research applications as described herein). These biomarkers may be used to reduce the cost and increase the efficiency and sensitivity of medical and research techniques. In one embodiment, one or more biomarkers or methods of using the biomarkers may be marketed to medical or research customers or potential customers. In one embodiment, a fee-based service may be provided to medical or research organizations wherein information relating to a medical image is obtained and analyzed for the presence of one or more biomarkers and the resulting information is returned in exchange for a fee. The amount of the fee may be determined, at least in part, by the type of image information that is provided, the type and degree of analysis that is requested, and the format and timing of the analysis. It should be understood that aspects of the invention may be applicable to image information obtained from one or more of many different scanning modalities (including, but not limited to, micro CT, MDCT, rotational angiography, MRI, PACS). This information may be received from many different sources, including, but not limited to one or more of the following: medical centers, large pharmaceutical companies (e.g., in association with pre-clinical evaluations or during clinical trials), CROs (for both pre-clinical and clinical analyses), medical laboratories and practices (e.g., scanning centers), hospitals, clinics, medical centers, small biotechnology companies (e.g., in association with pre-clinical evaluations or during clinical trials), and biomedical research organizations. The results of the analysis then may be returned to any one of these organizations. In some embodiments, the analysis results may be returned to the same entity that sent the image information. In other embodiments, the results may be returned to a different entity (e.g., the image information may be received from a scanning laboratory and the analysis may be returned to a physician). One or more steps involved with receiving the information, analyzing the structural features, processing the results and forwarding the results to a recipient may be automated. It also should be appreciated that one or more of these steps may be performed outside the United States of America. Business procedures (e.g., marketing, selling, licensing) may be performed individually or collaboratively.

Aspects of the invention may be described herein in the context of individual analytical steps, particular structural features, etc. However, it should be appreciated that any of the methods and devices described herein also may be incorporated into a business method associated with the use of a biomarker based on one or more blood vessel structural features or patterns (e.g., structural features or changes observed in vascular casts obtained from therapeutic and/or disease models or conditions).

Aspects of the invention may be automated (e.g., using one or more computer-implemented acts described herein). It should be appreciated that one or more pattern parameters (e.g., individual blood vessel structural feature(s), distributions of blood vessels or blood vessel structural features, or combinations thereof) may be analyzed using one or more quantitative and/or qualitative methods (e.g., based on binned data). In some embodiments, one or more parameters may be measured and quantified and the measurements may be analyzed using standard quantitative and/or statistical techniques for evaluation and/or comparison with threshold or reference values as described herein. In certain embodiments, one or more parameters may be evaluated using a predetermined scoring method, for example based on predetermined factors (e.g., for binned data). Geometrical parameters may be represented using vectors. For example, a distribution of blood vessels, blood vessel curvatures, blood vessel tortuosity, or blood vessel directions within a volume of interest may be represented using a plurality of vectors. Separate vectors may be used to represent separate vessels (e.g., vessels for which a connectivity has not been determined during the analysis). However, separate vectors also may be used to represent individual segments or fragments of a single blood vessel or portion of a vascular tree (e.g., for which connectivity has been or may be determined during the analysis). Vasculature pattern parameters may be analyzed using any appropriate technique for separating and/or categorizing numerical values or scores.

In some embodiments, a score may be obtained to relate a pattern parameter to the probability of a physiological condition such as a disease or a stage of a disease. Aspects of the invention can be used for in situ diagnostic, interventional and therapeutic analysis of one or more disease loci associated with aberrant internal structures. As used herein “in situ” means in an animal (e.g., a human) body as opposed to in a biopsy or other tissue sample. Aspects of the invention can be used to research structural changes associated with a disease, for developing and evaluating disease treatments including therapeutic drugs, and for other purposes. Aspects of the invention include automatically analyzing a structural feature or pattern and automatically generating a score based on the analysis.

In some embodiments, aspects of the invention include detecting and/or analyzing selected internal tubular networks in situ in animals and/or in vascular casts. As used herein, an internal tubular network means a network of connected cylindrical internal body structures. Tubular networks include, but are not limited to, cardio-vascular, respiratory, gastrointestinal, and genito-urinary systems and portions thereof within animal bodies. Accordingly, the cylindrical structures may include branched, straight, curved, and/or twisted cylindrical elements. The cylindrical structures and elements may include not only cylinders, but also may include flattened or otherwise distorted regions. The cross-section of a cylindrical structure or element may be circular, oval, approximately circular, approximately oval, or more irregular in nature. The internal diameter of the cylindrical elements may vary or may be approximately the same over the region of interest. A tubular network such as a circulatory network may be closed off from the environment outside the animal. In contrast, tubular networks such as respiratory and gastrointestinal networks may be open to the outside environment. In some embodiments, appropriate casting and/or contrast agents (e.g., inhaled agents) may be used to analyze respiratory and/or gastrointestinal networks.

In one embodiment, aspects of the invention include analyzing a segmented tubular network (e.g., a segmented vascular network). In one embodiment, a segmented representation of a network, or a portion thereof, may be obtained (e.g., from an existing database or a remote site) and analyzed. In another embodiment, a segmented representation of a network, or a portion thereof, may be generated from structural data and then analyzed. According to aspects of the invention, an analysis may include detecting the presence or absence of one or more structural features or patterns, measuring or evaluating the extent of one or more structural features or patterns, or a combination thereof.

In one embodiment, aspects of the invention are useful for selectively detecting and/or analyzing patterns (e.g., structures) of an animal's vasculature to detect or monitor one or more blood vessel patterns (e.g., structures) that may be indicative of a physiological condition of the animal. A structural pattern or feature may be detected and/or analyzed for blood vessels of any size including, but not limited to, arteries, arterioles, veins, venules, and capillaries.

In one embodiment, aspects of the invention are useful for selectively detecting and/or analyzing structural features or patterns of an animal's vasculature to detect or monitor one or more blood vessel structures that are characteristic of disease (e.g., a disease associated with angiogenesis). A blood vessel structure or pattern characteristic of a disease (e.g., a disease associated with angiogenesis) may provide an early diagnostic indication of the presence of the, which can allow for early treatment that can improve a patient's prognosis. In other embodiments, a blood vessel structure or pattern characteristic of a disease (e.g., a disease associated with angiogenesis) can be used as a marker (e.g., a biomarker) for staging and/or grading, to monitor disease progression, evaluate a prescribed therapy, and/or identify and/or validate a drug or treatment regimen for the disease. Diseases associated with abnormal vasculature structures or patterns include, but are not limited to, cancer, cardiovascular, dermatologic (skin), arthritic, musculoskeletal, central nervous system, neurologic, pulmonary, renal, gastrointestinal, gynecologic, genitourinary, inflammatory, infectious, and immunologic diseases.

A cancer may be a solid tumor or a leukemia. When the cancer is a leukemia, methods of the invention may be directed to detecting and/or analyzing vasculature pattern(s) in the bone marrow of an animal (e.g., human).

It also should be appreciated that aspects of the invention may include performing any combination of two or more acts described herein and that certain acts may be omitted in some embodiments. In one embodiment, the presence of one or more structural abnormalities may be identified or detected in a body region without generating and/or analyzing a structural representation of that body region. For example, the presence of a blood vessel abnormality may be detected directly from structure data for a body region without generating a structural representation of the vasculature for that entire body region. In another embodiment, an analysis may involve selectively representing one or more abnormal structures if they are present in a body region without representing normal structures in that body region (e.g., abnormal blood vessel structures may be represented without representing any normal blood vessels, or without representing all the normal blood vessels, without representing most of the normal blood vessels, etc.). In another embodiment, an abnormal vascular structure may be identified or detected without obtaining a detailed representation of the all the blood vessels in a body region. It may be sufficient to detect the presence of or outline of a vascular tree in a body region and perform an analysis that identifies or detects abnormal structures on specific blood vessels or the presence of excessive vascularization (e.g., a clump of neovasculature representing malignancy) without representing all the normal details of the vascular tree or even detecting individual blood vessels in the vascular tree. Accordingly, in some aspects a low resolution data set for a body region may be sufficient to detect or identify certain structural indicia of a disease such as cancer.

Aspects of the invention may include automating one or more acts. For example, an analysis may be automated in order to generate an output automatically. Acts of the invention may be automate using, for example, a computer system.

As should be appreciated from the foregoing, in one embodiment, raw or processed structure data may be obtained at a medical or research center and sent to a computer at a remote site where one or more of the analytical steps described above may be performed (e.g., for a fee). The output from the analysis may be then returned to the medical or research center either in computer readable form to a computer at the medical or research center, in a hard copy, in another tangible form, or in any other suitable form including those described herein.

In another embodiment, one or more software programs that implement one or more functionalities described herein may be provided and installed at a medical or research center (e.g., for a fee). The programs can be provided on disk, downloaded from an internal or remote (e.g., external) site, or loaded in any suitable manner. Reference information that is used in any functionality described herein may be provided along with the software or separately. In one embodiment, reference information (e.g., information relating to normal or abnormal blood vessel structures) may be available on disk, downloaded from an internal or remote (e.g., external) site, or loaded in any suitable manner.

As used herein, “remote” means at a site that is different from the immediate location of the imaging device (e.g., the medical scanner). The remote site can be a central computer or computing facility at a hospital, medical, or research center (e.g., within the network or intranet of the center), or can be outside the hospital, medical, or research center (e.g., outside the network or intranet of the center). The remote site can be in the same state, in a different state, or in a different country from the site of data acquisition by the imaging device.

In some embodiments, multimodal analyses (e.g., using structure data from two or more different types of imaging devices) may be used together. Accordingly, aspects of the present invention may include the ability to process and analyze different types of structure data and either combine the results to generate a combined output, or to generate a separate output is generated for each imaging modality. In some embodiments, an organ, tissue, or animal perfused with a casting agent and/or an imaging agent may be sent to an imaging center for analysis.

In some embodiments, in vivo and/or ex vivo casting methods of the invention can be used to identify one or more vascular patterns (e.g., including one or more structural parameters, structure distributions, combinations thereof) and/or time-dependent changes thereof that can be used as biomarker(s) for a disease or a response to a therapy, or for monitoring patients for indicia of disease or response to therapy, or for other applications where vascular information may be informative. Accordingly, such vascular patterns or changes thereof identified according to methods of the invention can be used for diagnostic, interventional, therapeutic, research, and treatment development and evaluation. Non-limiting examples of some of these embodiments are described below.

Diagnostic Applications

In one embodiment, aspects of the invention can be used to detect and diagnose diseases associated with patterns (e.g., individual structural features or distributions) of in situ tubular networks. In some embodiments, patterns or structures that are used as markers for in vivo detection or diagnosis may have been identified by analyzing vascular casts (e.g., of disease models). In some cases, a diagnosis can be rendered from an examination of the patterns (e.g., individual structural features or distributions) of interest at a single time. Alternatively, disease progression in a subject can be tracked by performing a structural analysis at two or more time points. Disease tracking can be used to provide diagnostic and prognostic information for a patient. For example, disease progression information can be used to assess the aggressiveness and/or invasiveness of a tumor.

The invention can be used to screen an individual or a population for the presence of indicia relating to one or more diseases. As mentioned above, the screen may be a whole body screen, or may be focused on one or more target regions (e.g., specific organs or tissues).

In one embodiment, the techniques described herein can be used automatically to identify individuals with one or more disease-associated structural patterns or features. These individuals can be subsequently tested for additional indicia of disease. The subsequent testing can take any suitable form, as the aspects of the present invention described herein are not limited in this respect. For example, follow on testing can employ conventional techniques. As a non-limiting example, the use of aspects of the present invention may enable cost-effective screening techniques that may identify a relatively small pool of candidates as at risk of a disease, and may justify the use of relatively more expensive testing procedures to reach a final diagnosis or prognosis, wherein the follow on techniques may be too expensive to administer to a wider sample that has not been narrowed using the techniques of the present invention described herein. As a further example, aspects of the present invention described herein, either alone or in combination with other techniques, can be used to perform subsequent tests. In this respect, the sensitivity of the initial screening can be set relatively high, such that it may indicate some false positives, and subsequent application of techniques in accordance with aspects of the present invention described herein can be employed with a higher degree of sensitivity that may provide more detailed information.

In one embodiment, aspects of the present invention can be used to screen a population of at risk individuals (e.g., individuals with genetic or other risk factors for a disease such as cancer, a circulatory disorder, or other disease) to identify the presence of disease indicia in one or more individuals.

In one embodiment, diagnostic methods of the invention are computer-implemented to increase efficiency and throughput, and reduce variability associated with individual physicians. However, as discussed herein, in some embodiments, the final diagnosis may be made by a physician based on information generated by an automated analysis or a structural representation using aspects of the invention described herein.

As shall be appreciated from the foregoing, aspects of the invention can be used on patients known to have a disease, or can be used to screen healthy subjects on a regular basis. A subject can be screened for one or more diseases. Screening can be done on a regular basis (e.g., weekly, monthly, annually, or other time interval); or as a one time event. Different conditions can be screened for at different time intervals and in function of different risk factors (e.g., age, weight, gender, history of smoking, family history, genetic risks, exposure to toxins and/or carcinogens etc., or a combination thereof).

In one embodiment, aspects of the invention can be employed to diagnose, evaluate or stage diseases associated with changes in vasculature structure. The detection of small changes in vasculature structure may be informative for early stage disease detection and disease monitoring. A high-resolution three-dimensional image of a vasculature structure may be analyzed and one or more patterns (e.g., individual structural features or distributions) may be evaluated for the presence of abnormal properties. In one embodiment, a vasculature structure may be a vascular tree including a series of interconnected branched blood vessels and may include arteries, arterioles, veins, venules, capillaries, and other sized blood vessels. According to aspects of the invention, different sizes of blood vessels can be detected and represented. In some aspects of the invention, the vascular tree of the entire body can be analyzed, and in other aspects the vascular tree of a target organ, tissue, or part thereof can be analyzed. In some aspects of the invention, a vascular tree containing only a subset of blood vessel sizes is analyzed (e.g., blood vessels with a diameter below about 500 microns, preferably below about 200 microns, more preferably below 100 microns, even more preferably below 50 microns, and even more preferably below 25 microns). In one embodiment, only capillary blood vessels are analyzed. In another embodiment, capillaries and small arteries and veins (e.g., arterioles and venules) are analyzed. For example, an arborescent vasculature can be analyzed in any tissue where it is found (e.g., an arborescent mucosal vasculature such as the oesophageal arborescent mucosal vasculature).

The branches of a vascular tree may be analyzed in a vascular cast or in vivo in a subject to glean information about the status of a patient. In one embodiment, the branches of a vascular tree may be followed to identify specific regions where certain characteristics of angiogenesis may be evaluated (e.g., start with a large branch and follow the tree to second, third, or fourth, or subsequent levels of branching to identify small blood vessels that may have abnormal structures if they are providing a blood supply associated with a disease). Alternatively, several different blood vessel sizes in the vascular tree may be evaluated for signs of angiogenesis. In another embodiment, the overall branching pattern of a vascular tree can be analyzed. For example, a healthy vascular tree may be approximately hierarchical in that the size of the blood vessels generally decreases as the vessels branch. In contrast, a diseased (e.g., angiogenic) vascular tree may be less hierarchical with areas of significant blood vessel branching with little or no decrease in blood vessel size. It should be appreciated that the nature and extent of the analysis may depend on the goal of the diagnostic evaluation. For example, a full body scan can be evaluated selecting all vascular structures and analyzing the entire vascular network for signs of different diseases. Alternatively, a region of a body suspected of being diseased may be selected and the data may be processed to focus on the vasculature in that region (e.g., to obtain a segmented representation of structures in the region of interest). A region of interest may be an organ (e.g., pancreas, liver, breast, colon, etc.) or a tissue (e.g., skin epidermal tissue). The presence of an abnormal vasculature structure can be an early indication of a range of diseases for which early detection is critical for effective treatment.

Diseases associated with changes in vascular structure (e.g., that can be detected by the presence of abnormal vascular patterns at a given time or abnormal structural changes observed as a function of time) include, but are not limited to, cancer, heart diseases and related circulatory disorders, eye diseases, skin disorders, and surgical conditions. For example, diseases and conditions associated with changes in vascular structure include, but are not limited to, tumor angiogenesis, recurrent and progressive cancers, coronary artery disease, cardiomyopathy, myocardial ischemia, arteriosclerosis, atherosclerosis, atherosclerotic plaque neovascularization, arterial occlusive disease, ischemia, ischemic or post-myocardial ischemia revascularization, peripheral vascular disease (including diabetic retinopathy), thromboembolic diseases (e.g., stroke, pulmonary embolism, brain aneurisms, and deep venous thrombosis), claudication, rheumatologic disorders (e.g., arthritis), immune disorders (e.g., rheumatoid arthritis, vasculitis, Wegner's granulomatosis, and systemic lupus erythematosis (SLE)), pulmonary disorders (including, emphysema, COPD, idiopathic pulmonary fibrosis, pulmonary arterial hypertension, and other respiratory disorders), myeloma, vascular proliferative disorders, gastrointestinal disorders (e.g., Crohn's disease, ulcerative colitis, and inflammatory bowel disease (IBD)), gynecologic disorders (endometrial polyp, vaginal bleeding, endometriosis, dysfunctional uterine bleeding, ovarian hyperstimulation syndrome, preeclempsia, polycystic ovarian syndrome (PCO), cervical cancer, and cervical dysplasia), skin disorders (infantile hemangioma, verruca vulgaris, psoriasis, neurofibromatosis, epidermolysis bullosa, Stevens-Johnson syndrome, and toxic epidermal necrolysis (TEN)), eye disorders (macular degeneration, maculopathies, diabetic retinopathy, and retinopathy of prematurity (retrolental fibroplasia)) wound healing, inflammation associated with immune responses, ischemia including limb ischemia and cardiac ischemia, Alzheimer's disease and other disorders such as wound dehiscence, Buerger Disease (thromboangitis obliterans, arteriosclerosis obliterans (ASO), ischemic ulcers) multiple sclerosis, idiopathic pulmonary fibrosis, HIV infections, plantar fasciosis, plantar fasciitis, Von Hippel-Lindau Disease, CNS hemangioblastoma, retinal hemangioblastoma, thyroiditis, benign prostatic hypertrophy, glomerulonephritis, ectopic bone formation, and keloids.

These different diseases are characterized by different changes in vasculature structure. Accordingly, in one aspect of the invention, parameters and scoring methodologies are used to detect, diagnose, and monitor particular diseases and their related therapies based upon particular characteristics of vasculature structure indicative of the disease (e.g., one or more characteristics identified from the analysis of a vascular cast of a disease model). Even within each disease category, different diseases can be characterized by different changes in vasculature structure. Accordingly, structure mining and scoring can be fine-tuned to increase the sensitivity for particular types of disease within a category (e.g., lung cancer score, breast cancer score, etc., can be developed). Patient-specific scoring parameters can also be developed to follow the progression of a specific disease or disorder in a patient.

Structural vasculature changes include changes in vascular architecture and vascular morphology affecting blood vessels and/or lymph vessels. Structural changes can involve neovascularization (including the growth of large blood vessels (e.g., arteriogenesis) and the growth of microvasculature (angiogenesis)), large blood vessel expansion, and vascular necrosis. Angiogenesis involves the formation of new blood vessels that sprout from preexisting blood vessels. Angiogenesis is different from vasculogenesis, which is the de novo formation of vessels that occurs primarily during development. Vasculogenesis is rarely associated with a disease or disorder. However, aspects of the invention can be used to study the natural process of vasculogenesis to help identify and understand defects in de novo blood vessel formation.

Angiogenesis is often associated with tumor growth and is a useful biomarker for cancer. Angiogenesis also can be associated with conditions where new blood vessel growth occurs in response to a reduced oxygen supply or blood flow (whether due to thrombosis, embolism, atherosclerosis, or other chronic occlusion or narrowing of the vasculature). Certain respiratory, cardiovascular, and inflammatory disorders also are associated with angiogenesis.

Angiogenic blood vessels have structural characteristics that are different from those of established blood vessels. For example, the branching patterns and tortuosity of angiogenic blood vessels are very different from those of normal blood vessels. These and other structural features are found predominantly in microvasculature and can be used for mining and scoring vasculature structural images. However, changes in larger blood vessels such as arteries and veins also may be associated with certain diseases or disease stages (e.g., growth and development of large tumors or late-stage tumors).

The vasculature that supports a tumor is typically associated with the connective tissue of the tumor (the stroma) that supports the malignant cells (in the parenchyma). A discussed above, tumor blood vessels are irregularly spaced and characterized by heterogeneous structural patterns or features. However, the formation of tumor blood vessels and other forms of angiogenesis may involve a series of characteristic stages (see, for example, Dvorak, 2003, American Journal of Pathology, Vol. 162:6, pp. 1747-1757, the disclosure of which is incorporated herein by reference in its entirety). Early stage angiogenesis may be characterized by vascular hyper-permeability, fibrin deposition and gel formation, and edema. This may result in the enlargement of micro-vessels such as venules. The cross-sectional area of an enlarged micro-vessel may be about 4 fold that of a normal micro-vessel. The perimeter of an enlarged micro-vessel may be about 2 fold that of a normal micro-vessel. Enlarged micro-vessels may occupy about 4-7 fold the volume of normal micro-vessels in a region of active angiogenesis. The appearance of enlarged micro-vessels may be followed by the appearance of “mother” vessels that are enlarged, thin-walled, serpentine, and hyper-permeable. Mother vessels may undergo a process of bridging whereby trans-luminal bridges are formed dividing the blood flow within the vessel into smaller channels. A developing mother vessel also may contain one or more glomerular bodies that may expand to divide the lumen of the mother vessel into several smaller channels that are typically tortuous. Bridging and glomerular body formation in mother vessels may lead to the appearance of small capillaries characteristic of angiogenesis. However, certain mother vessels persist as abnormally enlarged vessels with thin walls. These vascular malformations are often characterized by the presence of an asymmetric muscular coat and perivascular fibrosis. Small arteries and arterioles also may increase in size in diseased tissue. Aspects of the invention include detecting and/or monitoring any one or more of the blood vessel structural changes described herein. In one embodiment, the presence of one or more patterns (e.g., individual structural features or distributions) characteristic of new blood vessel formation may be used to detect or monitor a disease. In another embodiment, the presence of one or more specific patterns (e.g., individual structural features or distributions) may be used to determine the stage of angiogenesis (e.g., early-stage, mid-stage, late-stage, etc.) in a body region. In some embodiments, one or more of such characteristic patterns or structures may be identified form the analysis of appropriate vascular casts (e.g., of diseased tissue or organs, for example, in an animal model).

Accordingly, abnormal changes in blood vessel size (diameter and/or length) can be early signs of diseases such as cancer or other disease associated with an increased blood supply. Changes in blood vessel size may occur before any structural signs of angiogenesis appear. In one embodiment, aspects of the invention are useful to detect blood vessels (e.g., capillaries) that are swollen and/or longer than normal. For example, aspects of the invention are useful to detect abnormally long intrapapillary capillary loops in situ (e.g., associated with early stages of cancer in oesophageal mucosa).

In some embodiments, blood vessel changes indicative of necrosis in tumor tissues may be indicative of the aggressiveness of the tumor tissue and/or the likelihood of metastasis, and/or the responsiveness to therapy, and/or the efficacy of a therapeutic treatment (e.g., a candidate drug), and/or an therapeutic treatment selection and/or modification (e.g., a change in drug or dose for an individual patient). Accordingly, in situ patterns (e.g., individual structural features or distributions) indicative of necrosis may be useful biomarkers for patient prognosis. In certain embodiments, necrosis within a region of a tumor may be indicated by one or more of the following patterns (e.g., individual structural features or distributions) within that region: a collapse in blood vessel structure, poor vascularization (e.g., a low blood vessel density relative to other regions of the tumor or relative to the perimeter of the tumor), a change in blood vessel size or shape over time, a lower than threshold number of blood vessels, blood vessels (e.g., in the microvasculature or the capillaries) that are separated by a greater than threshold distance (e.g., by more than 100 microns, more than 150 microns, or more than 200 microns) within a volume of the tumor, micro-vessel diameter and/or density indicative of undervascularization, etc., or any combination thereof. In some embodiments, a volume of avascularization or undervascularization may be evaluated or quantified and used as an indicator of necrosis. It should be appreciated that other indicia of necrosis may be used, alone or in combination with blood vessel features. Other indicia may include indicia of tissue collapse or cavitation that may be visualized (e.g., using CT etc.) and/or indicia of tissue viability using one or more markers of metabolic activity (e.g., ones that may be analyzed using a PET scan, etc.). One or more reference indicia (e.g., a reference volume of avascularization or undervascularization may be identified by analyzing vascular casts of necrotic tumor tissue (e.g., in a xenograft tumor model, for example in an orthotopic or an ectopic tumor xenograft).

Aspects of the invention may be used for the detection (e.g., the automatic detection) of necrotic areas in a subject (e.g., in a tumor in a subject). A necrotic region is an avascular region within the boundary of a diseased tissue. Methods of the invention may be used to detect (e.g., automatically) the transition between the vascularized diseased tissue and avascular region that defines the boundary of the necrotic region.

Aspects of the invention also may be used to detect or evaluate (e.g., automatically) a response to therapy. For example, a response to therapy (e.g., to a specific drug and/or a specific dosage of a drug, and/or to a combination of drugs and specific dosages of these drugs, etc.) can be detected and assessed as follows. Changes in the vascular patterns (e.g. vessel normalization/straightening, disappearance of smaller diameter vessels leading to lower micro-vessel density and to skewing of the vessel diameter distribution towards the larger vessels) may be detected and/or evaluated within the volume defined by the boundary of the diseased tissue and the boundary of the necrotic area. An increase in the absolute volume size of the necrotic area and/or the rate of such change while the total volume of the disease (e.g. tumor) volume stays constant may be detected and/or evaluated as an indicator that the therapy is effective. An increase in the ratio between the absolute volume size of the necrotic area and the total disease (e.g., tumor) volume and/or the rate of change in this ratio may be detected and/or evaluated and used as an indicator that the therapy is effective. A ratio of the diseased tissue volume and the necrotic region volume may be detected and/or evaluated and when it approaches 1 and the overall diseased tissue volume starts shrinking it provides an indication that a therapy is effective. Accordingly, reference indicia may be obtained from analyzing casts (e.g., appropriate vascular casts).

Structural representations of blood vessels can be mined to identify and evaluate certain patterns (e.g., individual structural features or distributions) that can be used to provide a score that is related to the probability that the blood vessels are normal or abnormal (e.g., disease associated). Patterns (e.g., individual structural features or distributions) for scoring blood vessels include, but are not limited to, the following: diameter, curvature, tortuosity (including, for example, the degree of tortuosity, the length of the blood vessel along which abnormal tortuosity is observed, etc.), variability or heterogeneity (including spatial variability or heterogeneity over distance or in a volume), branching shape or pattern, branching density, branching hierarchy, blood vessel density, distribution of vessel size (ratio of microvasculature to macrovasculature) a field effect (the presence of blood vessels bending towards a specific region), blood vessel diameter distribution, variability of the geometric orientation of blood vessels or fragments thereof, and the distribution of the orientation(s) within a field. The score may have more significance if two or more of these parameters are evaluated. In some embodiments, a score is generated using one or more of these structural parameters combined with additional information such as patient-specific medical information (e.g., age, weight, height, gender, etc.) and the presence of one or more additional indicators of disease such as a visible lesion on an X-ray or other image. In some embodiments, a score can be provided for a tumor. An example of a useful score is one that reflects the vascularity of a tumor. An abnormally high vascularity (measured as a higher than normal blood vessel number, density, length, or combination of the above) is generally indicative of a more aggressive or invasive tumor. In one embodiment, vascularity is evaluated by measuring the volume of the lumen of angiogenic vasculature (the volume within the blood vessel tree associated with a tumor). In another embodiment, a measure of vascularity is provided by dividing the volume of the angiogenic lumen by the volume of the solid tumor. Additional information can be gleaned from obtaining a score (or other structural evaluation) at two or more times. A changing score (or other structural evaluation) is indicative of an evolving vasculature that could be associated with a disease or disorder. It should be appreciated that the patterns (e.g., individual structural features or distributions) described herein can be identified and analyzed for a field of analysis without imposing a connectivity on the vessels being studied. In some embodiments, it may be sufficient to analyze only fragments of blood vessels in order to detect one or more structural features of individual vessels or geometrical features of a field of vessels that are different from normal features. For example, blood vessel fragments having an average length of 0.5 mm, 1 mm, 5 mm, 10 mm, 50 mm, 1 cm, 5 cm, 10 cm, 50 cm, etc. may be used. However, it should be appreciated that shorter or longer or intermediate lengths may be used.

The scoring and mining aspects of the invention described herein can be automated. Accordingly, diseased (e.g., angiogenic) vasculature can be automatically detected amidst normal vasculature. Various vasculature parameters can be automatically detected and scored, either separately or in any combination, including vessel tortuosity, vessel branching, vessel density, and total intra-vascular volume, but the invention is not limited to any particular parameter or combination.

In one embodiment, aspects of the invention can be used to detect blocked blood vessels, and thromboembolic events, including stroke, lung emboli, blocked micro-coronaries, deep-vein thrombosis, etc. Blocked blood vessels can be detected (1) directly by detecting structural changes in the blocked blood vessel (e.g., detecting a clot, wall thickening, or other signs of reduced flow) and/or (2) indirectly by detecting new vasculature that was generated in response to the blockage. In general, the formation of collateral blood vessels is more ordered than angiogenesis associated with cancer. One aspect of the invention described herein also allows clots to be detected in small blood vessels.

As discussed above, aspects of the invention can be used to screen the entire vasculature structure of a human or other animal to screen for any form of abnormality in any tissue. Alternatively, a subset of the body may be screened. Accordingly, vasculature structures such as a vascular tree can be analyzed for one or more organs or tissue types. In addition, only a portion of the vasculature may be analyzed within any target volume as opposed to the entire vascular tree in that volume. This may be done by analyzing structure data focused on the area of interest, or large amounts of structure data may be obtained, but an analysis may be restricted to a subset of the available data. In some embodiments, only a portion of a vascular tree may be represented and/or analyzed, for example only those vessels that are of a particular size. In other embodiments, only fragments of a vascular tree are represented and/or analyzed if the fragments are sufficiently informative to provide patterns (e.g., individual structural features or distributions) of interest. Fragments may include branches or may be unbranched. The portion of the vasculature being analyzed may be statistically significant, such that any observation (normal or abnormal) is physiologically significant. For example, branched structures may not be required for the analysis if a sufficient number of vessel substructures are analyzed to confidently detect any other patterns (e.g., individual structural features or distributions) that may be associated with vasculature changes (e.g., angiogenesis) such as high vessel density. In aspects of the invention, vascular patterns may be detected and/or evaluated in situ in a volume of 1 mm³, 2 mm³, 5 mm³, 1 cm³, 2 cm³, 5 cm³, 10 cm³, etc. However, smaller or larger or intermediate volumes also may be analyzed. In some embodiments, vascular patterns or structures are evaluated over an entire model tissue or organ (e.g., for an entire orthotopic or ectopic tumor model).

Different tissues and organs have different and characteristic blood vessel patterns (e.g., the lung which is highly vascularized). Accordingly, in one embodiment, structural analyses and associated structural parameters may be optimized for evaluating different tissues. For example, reference biomarkers identified using vascular casts can be tumor-specific and/or tissue-specific markers (e.g., specific markers of cancer for the lung, colon, liver, kidney, pancreas, throat, etc., or a combination of two or more thereof).

In some embodiments, scan data is obtained and/or analyzed for one or more organs (e.g., lung, heart, colon, brain, liver, pancreas, kidney, breast, prostate, etc.) or tissue (e.g., skin, bone, etc.) or portion of any of the above.

Brains may be evaluated for signs of brain tumors and/or other neurological disorders that can be associated with changes in vascular patterns. For example, Alzheimer's may be associated with certain vascular abnormalities. In one embodiment, one or more changes in blood vessel pattern (e.g., shape and/or size) may be detected as an indicator of high blood pressure in the brain.

In some embodiments, certain specific regions of organs or tissues are focused on. For example, atherosclerosis is typically found in certain parts of the arterial tree (e.g., bifurcations, side branches, regions opposite flow dividers, and other areas where angiogenesis often occurs in association with atherosclerosis) and certain cancers tend to occur more frequently in certain organ or tissue regions (e.g., colon cancers are not distributed evenly along the length of the colon).

In other embodiments, aspects of the present invention may be used to follow up with individuals who have been identified as having one or more other indicia of disease (e.g., fecal occult blood, a colon polyp, a lung nodule, one or more cysts or other indicia of disease). Aspects of the invention may be used to confirm the presence of a disease, determine a location for the disease-associated lesion, or provide an evaluation or prognosis of a disease. For example, aspects of the invention may be used to determine whether abnormal vasculature is present at the site of a lesion (e.g., a colon polyp, a lung nodule, a bladder cyst, a prostate cyst, a breast cyst, a spot on a mammography, or any other cyst, lump, or spot that may be detected physically, visually, or using any other diagnostic technique) and help evaluate the likelihood of a malignancy (or other carcinogenic disease stage) associated with the lesion. Accordingly, aspects of the invention may be used for virtual malignancy detection (e.g., virtual colonoscopy, virtual colon malignancy detection, virtual bronchoscopy, virtual lung malignancy detection, virtual mammography, virtual cystoscopy, etc.).

In other embodiments, aspects of the invention may be used for screening a cancer patient to evaluate the extent of a cancerous lesion and/or to screen for the presence of one or more metastatic lesions (e.g., one or more loci associated with angiogenesis). A cancer patient may be screened upon initial diagnosis of a primary cancer. In addition or alternatively, a cancer patient may be screened at least once after an initial cancer treatment (e.g., surgery, radiation, and/or chemotherapy). This screening may include the original cancer locus to detect any cancer recurrence. This screening may include similar body tissue to screen for the presence of other lesions in the same tissue or organ (e.g., the entire colon may be screened when a cancerous lesion is detected in one region of the colon, the second breast may be screened when a cancerous lesion is detected in one breast, etc.). This screening also may be extended to the whole body or to one or more other loci suspected of containing a metastatic lesion. In one embodiment, a cancer patient may be screened several times after an initial cancer treatment (e.g., at time intervals of about 6 months, about 1 year, about 2 years, about 5 years, or at other time intervals).

In one embodiment, a follow up procedure may involve screening one or more organs or tissues for the presence of a metastatic lesion. Different cancers may have different characteristic patterns of metastasis. Accordingly, different target loci may be screened for different cancers. For example, metastatic breast cancer typically spreads to the lungs, the liver, bone, and/or the CNS. Therefore, one or more of these tissue types or organs may be screened after a patient is diagnosed with breast cancer. Similarly, other target loci may be screened after a patient is diagnosed with another cancer type. In some embodiments, the entire body of a cancer patient may be screened for indicia of metastasis.

In one aspect, an initial screen may be performed on an entire body, or an entire organ, using a low resolution representation and/or, for example, analyzing only one or two or a small number (e.g., less than five) pattern parameters in order to detect indicia of a disease. Subsequently, the presence and or nature of the disease may be diagnosed using a higher resolution representation and/or, for example, analyzing one or more additional pattern parameters or alternative pattern parameters than those that were analyzed for the initial detection.

In some embodiments, small changes in blood vessel distributions may be observed (for example as measured by a ratio between the number of blood vessels of two or more different sizes in a region of interest, for example, a tumor in an animal model) and used as a biomarker. Such biomarkers may represent early changes (e.g., early changes in tumor growth or response to therapy) that occur before later changes in tumor size and/or tumor morphology. It should be appreciated that some or all of the diagnostic aspects of the invention can be automated as described herein.

Interventional Applications

Aspects of the invention also can be used to identify the location of a disease by locating one or more structural abnormalities associated with the disease (e.g., based on structure characteristics identified in a vascular cast of a disease model). This information can be used to target a biopsy procedure or a treatment (e.g., a treatment with one or more toxic chemicals, radiation, heat, cold, small molecules, gene therapy, surgery, any other treatment, or a combination of two or more of the above) to the precise location of a disease lesion, or for any other purpose.

In one embodiment, an imaging device is connected to a computer that provides a real-time visual display of the disease lesion. In one embodiment, a real-time visual display may be an accurate model of a body region and lesion along with associated vasculature (as opposed to an actual image). This visual information can be used to guide a surgical instrument for a biopsy. Alternatively, the information can be used to guide an invasive (e.g., surgical removal or bypass) or non-invasive (e.g., radiation) treatment procedure to the site of the disease lesion (e.g., tumor or blood clot).

In one embodiment, aspects of the invention may be used to identify an area of tissue for treatment before the treatment is applied. For example, a treatment target region may be identified by detecting a boundary of chaotic blood vessel structures. The area may be assessed after treatment to confirm that the treatment was appropriately targeted. In one embodiment, a structure may be analyzed pre-operatively to identify the extent of tissue to be removed from a body region. In one embodiment, a body region may be analyzed post-operatively to determine whether any abnormal structures were missed. This may be used to confirm the success of a radiation treatment or a surgical removal of diseased tissue. Alternatively, this may be used to decide on further surgery and/or another form of treatment. In another embodiment, a disease boundary may be defined or depicted by the boundary of abnormal vasculature. A treatment (e.g., radiation therapy, surgery, etc.) may be guided by and/or restricted to a volume encompassed by the disease boundary.

In one embodiment, aspects of the invention can be used to evaluate the success of a surgical implant or transplant. For example, aspects of the invention can be used to evaluate the formation of new blood vessels after an organ or tissue transplant.

In another embodiment, the development of new blood vessels may be monitored after removal of tumor tissue or after a tumor biopsy, both of which may trigger angiogenesis and/or convert a dormant tumor into a malignant tumor.

It should be appreciated that some or all of the interventional aspects of the invention can be automated as described herein.

Therapeutic

Aspects of the invention also can be used to optimize a therapeutic treatment for a patient. The extent of disease progression or regression can be monitored in response to different treatment types or dosages, and an optimal treatment can be identified. The optimal treatment may change as the disease progresses. The effectiveness of the treatment over time can be monitored by analyzing changes in disease-associated patterns (e.g., individual structural features or distributions) using the aspects of the present invention described herein (e.g., by reference to characteristic structural features identified in vascular casts of appropriate animal or tissue models for a disease, condition, or therapeutic response of interest).

In one embodiment, a first therapy can be administered and its effectiveness on slowing, stopping, or reversing abnormal blood vessel growth can be monitored either irregularly or at certain time intervals (e.g., daily, weekly, monthly, or other time intervals). In some embodiments, if a first therapeutic regimen does not have a desired effect on disease progression, a second therapeutic regimen can be evaluated. Similarly, additional therapeutic regimens can be evaluated on a patient-by-patient basis. Additionally, the invention can be used to optimize a chosen therapeutic regimen (e.g., optimize dosage, timing, delivery, or other characteristic of a drug or other treatment) by monitoring the effect of minor therapeutic changes and using the conditions that appear to be most effective for the condition and the patient.

When looking at the therapeutic effectiveness of a treatment, disease-specific parameters may be monitored. Of course, all parameters can be obtained and only a subset reviewed. However, it may be more efficient to simply obtain (a representation of) only those parameters that characterize the disease.

According to aspects of the invention, patterns (e.g., individual structural features or distributions) that are used to detect angiogenic vasculature and other abnormal blood vessels also can be used to monitor a disease response to treatment. For example, the total vascularity or any other volumetric analysis of angiogenic or other diseased vasculature, and the distribution of vessel size (e.g., a ratio of small to large blood vessels) can be used independently or together as indicators of disease progression or regression. In general, microvasculature disappears before macrovasculature if an anti-angiogenic treatment (or other disease treatment) is effective. Therefore, an effective treatment results in a shift in the distribution of blood vessel sizes towards larger vessels. An index of anti-angiogenic activity can be scored as either a loss of small blood vessels or a shift of observed blood vessels towards a single size (or both).

In another aspect, the parameters can be (or include) changes over time. For example, a structure present at a second time can be compared to a structure present at a first time. In one embodiment, a disease may be tracked pre-therapy and/or post-therapy. Naturally, additional time points can be used. The time points may depend on the condition being observed (e.g., is it the progression of a disease that is already identified, is it the screening of patient(s) over time). Time periods can be daily, weekly, monthly, annual, or shorter, intermediate or longer time periods. Time intervals may be a series of regular time periods. However, other time intervals may also be useful. In one embodiment, a patient-specific baseline is established and monitored over time. For example, vasculature changes in the colon, breast, or other tissue or organ can be monitored periodically.

In one aspect of the invention, a type of treatment may be determined by the degree or extent of abnormal vascular structures (e.g., angiogenesis) that is detected at one or more suspected disease loci (e.g., cancerous loci). For example, if a suspected cancerous locus or metastasis is pre-angiogenic or associated with early stage angiogenesis, it may be appropriate to monitor the locus without any form of treatment. However, an appropriate therapy may involve the administration of one or more angiogenesis inhibitors to prevent the formation of any new vasculature. If a suspected cancerous locus or metastasis is associated with mid-stage angiogenesis, an appropriate therapy may be the administration of one or more angiogenesis inhibitors. A patient with mid-stage angiogenesis at a suspected locus also should be monitored so that any further blood vessel development can be treated more aggressively. If a suspected cancerous locus or metastasis is associated with late stage angiogenesis, an appropriate treatment may involve at least one or more of chemotherapy (e.g., cytotoxic chemotherapy and/or hormone-based chemotherapy), radiation, surgery, and/or treatment with one or more angiogenesis inhibitors. However, it should be appreciated that any of the above treatment options may be used to treat a patient with any one or more lesions associated with any degree of angiogenesis.

Examples of angiogenesis inhibitors, include but are not limited to, 2-methoxyestradiol (2-ME), AG3340, Angiostatin, Angiozyme, Antithrombin III, VEGF inhibitors (e.g., Anti-VEGF antibody), Batimastat, bevacizumab (avastatin), BMS-275291, CAI, 2C3, HuMV833 Canstatin, Captopril, Cartilage Derived Inhibitor (CDI), CC-5013, Celecoxib (CELEBREX®), COL-3, Combretastatin, Combretastatin A4 Phosphate, Dalteparin (FRAGIN®), EMD 121974 (Cilengitide), Endostatin, Erlotinib (TARCEVA®), gefitinib (Iressa), Genistein, Halofuginone Hydrobromide (TEMPOSTATIN™), Id1, Id3, IM862, imatinib mesylate, IMC-IC11 Inducible protein 10, Interferon-alpha, Interleukin 12, Lavendustin A, LY317615 or AE-941 (NEOVASTAT™), Marimastat, Maspin, Medroxpregesterone Acetate, Meth-1, Meth-2, Neovastat, Osteopontin cleaved product, PEX, Pigment epithelium growth factor (PEGF), Platelet factor 4, Prolactin fragment, Proliferin-related protein (PRP), PTK787/ZK 222584, ZD6474, Recombinant human platelet factor 4 (rPF4), Restin, Squalamine, SU5416, SU6668, SU11248 Suramin, Taxol, Tecogalan, Thalidomide, Thrombospondin, TNP-470, Troponinl, Vasostatin, VEG1, VEGF-Trap, and ZD6474.

Some embodiments may include a method of selecting a subject for treatment and/or selecting a treatment or a course of therapy based on the analysis of certain in situ vascular structures. A method may involve analyzing in situ vascular structure(s) in a human subject to obtain, for example, a score. The score may be compared to a control score (e.g., in an apparently healthy population, or from a vascular cast of a healthy tissue, organ, or animal model) or to a previous score from a previous analysis on the same subject. The treatment or the course of therapy may be based on such a comparison. In some embodiments, obtaining an analysis of vascular structures is repeated so as to monitor the human subject's response to therapy over time. In some embodiments of this aspect of the invention, the method further comprises measuring a second index of disease in the human subject wherein deciding on the treatment or course of therapy is also based upon the measurement of said second index.

In certain embodiments, patients having a tumor that is under-vascularized (e.g., one that shows signs of necrosis) may be selected for treatment with one or more anti-angiogenic compounds. Under-vascularized tumors may be identified as those that have a low density of blood vessels, or for which the blood vessel diameters are low (e.g., below a threshold number typical of vascularized tumors).

Aspects of the invention also may include monitoring the effectiveness of a therapy by monitoring the presence of blood vessel patterns or features over time. For example, the progressive loss of blood vessels in a tumor in response to treatment may be a sign that a therapy is effective. In contrast, the absence of any impact on vascularization may be an indicator that a treatment is not being effective in a patient and that an alternative therapy should be considered or used.

It should be appreciated that some or all of the therapeutic aspects of the invention can be automated as described herein.

Research

In one embodiment, aspects of the invention can be used to understand structural changes associated with biological processes of interest (e.g., disease development and progression). For example, an animal's vasculature can be analyzed (e.g., using an appropriate vascular cast) to identify additional patterns (e.g., individual structural features or distributions) that may be associated with wound healing or different diseases or different disease stages. These additional patterns (e.g., individual structural features or distributions) may be used in one of more of the diagnostic, interventional, therapeutic, and/or development aspects of the invention.

In one embodiment, aspects of the invention can be used to understand structural changes associated with medical procedures. For example, an animal's vasculature can be analyzed to identify changes associated with post-surgical wound healing or implant/transplant (including xenografts) growth or rejection.

It should be appreciated that some or all of the research aspects of the invention can be automated as described herein.

Development and Evaluation of New Treatments Including Drug Screening and Validation

In another embodiment, aspects of the invention can be used in screens of compound libraries or to validate candidate compounds for treating diseases associated with abnormal internal structures (e.g., abnormal tubular networks). Aspects of the invention allow efficient high throughput analyses of internal structural changes. These changes can act as surrogate markers (biomarkers) for certain diseases. As a result, the screening process can be automated to a large extent, and the time for obtaining results significantly shortened when compared to current validations that often involve waiting for disease symptoms to change and also may require tissue biopsies.

Surrogate markers: Aspects of the invention may be used for identifying and quantifying vascular patterns (e.g., structural features) that can be used as surrogate markers for diagnostic, therapeutic, and research and development purposes. Surrogate markers are useful for reducing the time of diagnosis, therapy evaluation, and drug development. A surrogate marker can be used as an early indicator for disease diagnosis, disease prognosis, or drug effectiveness, without waiting for a clinical outcome (e.g., increased survival time in response to a drug). So, a vasculature analysis can be used as a surrogate marker for drug development (in both pre-clinical and clinical trials), for clinical screening (e.g., breast, lung, or colon screening), and for clinical therapy monitoring. For example, vasculature structure is a useful surrogate marker for angiogenesis related diseases such as cancer.

In one embodiment, aspects of the invention provide methods for screening and/or validating candidate compounds or therapies for their effectiveness in treating neo-vasculature formation and/or vasculature pattern changes associated with disease. Aspects of the invention may be used to evaluate individual or small numbers of compounds or to screen libraries to evaluate and/or identify a plurality of candidate compounds (e.g., by administering these compounds, individually or in groups, to an experimental animal such as a mouse and evaluating their effect on angiogenic vasculature). Libraries may contain any number of compounds (e.g., from approximately 100 to approximately 1,000,000). Different types of compounds can be screened, including antibodies, small molecules, etc., or any combination thereof. However, the invention is not limited by the number and/or type of compounds that can be evaluated.

In one embodiment, the effectiveness of a candidate compound can be compared to a reference compound. A reference compound can be any compound with a known effect on a structure. For example, an angiogenesis inhibitor available from Genentech (South San Francisco, Calif.) as Avastin® is a known monoclonal antibody against vascular endothelial growth factor (VEGF) that can be used as a reference to test the relative effectiveness of a candidate compound on neovasculature growth. It should be appreciated that surrogate markers described herein may be identified from vascular casts as described herein.

In vivo models: According to aspects of the invention, compounds and therapies can be evaluated in the context of an in-vivo model such as an animal disease model. An animal disease model may be a transgenic animal, a recombinant animal, an orthotopic tumor model, an ectopic tumor model, or other suitable disease model. In some embodiments, an orthotopic or ectopic tumor model may be generated using a xenograft of tumor cells or tissue (e.g., of human tumor cells or tissue into a mouse or other non-human animal model). For example, a mouse with cancer or atherosclerosis can be used to evaluate, optimize, and identify useful therapies. Other animal models also can be used. Aspects of the invention may be useful for high-throughput analyses because they can detect small changes in vasculature and can be used to evaluate a therapy in a short time period with minimal manipulation since little or no invasive procedures are required.

Vascular analysis aspects of the invention can be used on an orthotopic model to test, for example, the effectiveness of a drug in a short period of time. For example, the effect of a candidate drug on angiogenesis in an orthotopic mouse tumor model may be quantifiable after about 5 days (e.g., between 1 and 10 days, depending on the model and the drug). In contrast, a subcutaneous cancer animal model requires approximately one month for tumor growth to be analyzed and compared to controls.

An orthotopic model can be used to model different diseases or clinical conditions. Examples include, cancer, tissue regeneration, wound healing (including healing after traumatic injury, healing after surgical intervention, healing of burnt tissue such as skin), tissue or organ transplant therapy, medical device implant therapy, other conditions associated with neovascularization or changes in normal vascular structure, or any combination of two or more of the above. However, the invention is not limited by the type of orthotopic model or the type of disease or clinical condition that is being analyzed.

A single orthotopic disease model animal may be useful for testing more than one candidate drug molecule since the analysis does not involve sacrificing the model animal. Accordingly, once a test with a first candidate is complete, a subsequent candidate can be evaluated in the same model animal. A series of candidates can be tested in a single model animal, with appropriate controls, provided the model retains features of neovascularization that are necessary for the assay.

It should be appreciated that some or all of the development aspects of the invention can be automated as described herein.

It also should be appreciated that any one or more structural parameters described herein may be evaluated by comparison to a reference parameter. In some embodiments, a reference parameter may be an amount or score for that parameter in a normal or healthy subject. In other embodiments, a reference may represent a diseased condition. A reference pattern or structural parameter may be identified from a vascular cast of healthy or diseased tissue (e.g., using a vascular cast of a suitable animal model). In some embodiments, a change or amount of any structural parameter that is correlated or associated with a disease or condition as described herein may be a statistically significant change or difference in that parameter in a diseased or test subject relative to a reference subject. In some embodiments, a difference or change in a structural parameter may be an increase or a decrease in a particular parameter (or a combination of parameters). An increase in a parameter may be at least a 5%, 10%, 20%, 30%, 40%, 50%, 60%, 70%, 80%, 90%, 100%, or greater increase in that parameter in a test subject relative to a reference subject. Similarly, a decrease in that parameter may be at least a 1%, 5%, 10%, 20%, 30%, 40%, 50%, 60%, 70%, 80%, 90%, or a 100% decrease of a measure of that parameter in a test subject relative to a reference subject. Once an amount of change or difference in a parameter has been correlated or associated with a disease or condition, that level may be used in subsequent methods according to the invention. Accordingly, in some embodiments, a difference of at least at least 1%, 5%, 10%, 20%, 30%, 40%, 50%, 60%, 70%, 80%, 90%, 100%, or more of any given structural parameter (e.g., tortuosity, density, volume, or any other individual structural feature or distribution of structures or structural features as described herein) relative to a reference value may be used as a threshold for methods of the invention. It should be appreciated that higher or lower or intermediate values may be used. It also should be appreciated that different parameters may have different threshold or reference levels. Also, different parameters (and/or different levels for each parameter) may be associated with different conditions or diseases. Accordingly, specific disease or condition values or thresholds may be identified for different parameters or combinations thereof. These threshold values may be used for disease detection, diagnosis, monitoring, or for any other therapeutic, clinical, or research application described herein (e.g., in automated methods described herein).

### EXAMPLES

### Example 1

### Examples of Diagnostic, Therapeutic, and Research Applications

The following example illustrates how aspects of the invention can be used for diagnostic, therapeutic, and research purposes by analyzing vascular structures associated with different diseases. However, it should be appreciated that the techniques described herein can be applied to different structures and for different diseases or conditions.

Bone analysis: Breast cancer often metastasizes to bone. However, there is currently no consensus on the optimal method for detecting a bone cancer lesion. In one embodiment, aspects of the invention can be used to diagnose a bone lesion and evaluate its response to treatment by analyzing blood vessel structures (and/or changes therein) in bones. A bone lesion can be of any type including osteolytic, osteoblastic, or a combination thereof. Lesions in the bone marrow can also be identified, diagnosed, and/or evaluated. Bone has a typical vasculature that is readily recognized. Using techniques described herein, changes in the vasculature and new vascular features can be distinguished from normal bone vasculature.

Certain conventional bone scan techniques such as PET use radio-labeled markers to identify cancerous tissue. However, such scans are complex and expensive, and are used only when there is a specific concern about the potential presence of a cancerous lesion in the bone of a patient. Aspects of the invention described herein do not require radio-labeled markers and provide structural information that may be easier to interpret and can be evaluated automatically. Bone vasculature analysis may be particularly useful for breast cancer patients to detect any early signs of cancer metastasis to bone loci. However, aspects of the invention may also be used to screen healthy subjects to detect any signs of vascular changes in their bones.

It should be appreciated that aspects of the invention also provide information that is useful for evaluating the stage of a bone cancer and for optimizing treatment for bone cancer.

Diabetic retinopathy: Diabetic retinopathy results from the formation of new blood vessels in patients with diabetes. Diabetic retinopathy causes retinal malfunction and visual complications leading progressively to blindness. If detected early, diabetic retinopathy can be treated or managed. For example, laser photocoagulation therapy can be used to prevent vision loss if blood vessel proliferation is detected early. In one embodiment, aspects of the invention can be used non-invasively to detect early blood vessel proliferation associated with diabetic retinopathy. The techniques described herein may enable the detection of earlier signs of neo-vascularization than methods such as fluorescein angiography or fundus photography. In addition, some embodiments of the invention do not require that a specialist be present at the same medical center as the patient, as detection and diagnosis may be performed at a remote location based on retinal blood vessel structural information derived from the patient.

Aspects of the invention also can be used to monitor and optimize therapeutic treatments to prevent or minimize vision loss in a diabetic patient. In particular, vascular structural information may be used to target a treatment to a region of the retina that is affected by early stages of diabetic retinopathy. The monitoring and treatment aspects also may be coordinated by a specialist at a remote location.

Lung Cancer: Lung cancer is a leading cause of cancer death, and early detection is the most effective technique for improving the chance of survival. Lung cancer shows up as pulmonary nodules on conventional two-dimensional chest radiographs and three-dimensional CT scans. However, aspects of the invention may be used to detect early changes in lung vasculature that appear before pulmonary nodules can be detected using conventional techniques.

In one embodiment, a subject's lung vasculature may be analyzed according to aspects of the invention to complement or confirm the diagnosis of a lung cancer that was initially detected using current chest X-ray or CT analytical techniques. The presence of abnormal vasculature at the same location as a spot on an X-ray may confirm the presence of a tumor at that site.

In another embodiment, aspects of the invention may be used as an initial screen to identify abnormal lung vasculature. It should be appreciated that if a pocket of angiogenic blood vessels is detected, follow up analyses may be performed using current chest X-ray or CT scan techniques. However, if the angiogenic blood vessels are detected early, cancer spots may not be visible using current non-invasive techniques. In one embodiment, a doctor may obtain a biopsy of the angiogenic region by inserting a bronchoscope through a subject's nose or mouth and down the throat to access the subject's airways and lungs and take a sample of the suspect tissue. Of course, alternative biopsy methods can be used. Biopsy techniques may be guided using aspects of the invention to make sure that a tissue sample containing abnormal vascular structures is removed. A suspect tissue sample can be analyzed in a laboratory, for example, to assay for the presence of one or more molecular indicators of cancer or other disease. However, in one embodiment, aspects of the invention provide a virtual biopsy that is sufficient to diagnose a condition without a tissue biopsy (e.g., a brochoscopy biopsy). In one embodiment, aspects of the invention may be used to monitor a lesion (e.g. by analyzing it at several time points separated by relatively small time increments such as hours, days, or weeks) in order to determine whether it is growing and malignant, without involving an invasive biopsy procedure.

In one embodiment, subjects at risk of lung cancer may be screened routinely for abnormal lung vasculature structures according to aspects of the invention described herein. Risks of lung cancer include, but are not limited to, smoking, pollution, and family history.

Chronic Obstructive Pulmonary Disease (COPD): COPD is a term that is used for two closely related diseases of the respiratory system: chronic bronchitis and emphysema. In many patients these diseases occur together, although there may be more symptoms of one than the other.

In one embodiment, aspects of the invention may be used to detect early signs of COPD/Emphysema early and to monitor the progress of the disease and its response to drugs and other therapies. Early signs of COPD/Emphysema include increased blood vessel growth in diseased lungs in response to hypoxia. These signs may be detected before symptoms such as a chronic cough and progressive heart and lung failure develop. Subjects at risk, including smokers and subjects with mild shortness of breath, may be screened routinely according to methods of the invention.

Pulmonary Embolism (PE): Pulmonary embolism can result from a blocked artery in a subject's lung. Every year, more than 600,000 Americans experience a pulmonary embolism with severe and often fatal consequences. In most cases, the blockage is caused by one or more blood clots that had traveled to the lungs from another part of the body.

According to aspects of the invention, one or more blood clots may be detected before they travel to a subject's lungs and cause severe damage. The most common sources of blood clots are the deep veins of the leg. A clot may break loose from a leg vein and travel to a pulmonary artery in the lung, where it can block blood flow and cause more severe problems than when the clot was in the leg vein. Smaller clots prevent adequate blood flow to the lungs, sometimes causing damage to lung tissue (infarction). Large clots that completely block blood flow can be fatal. Aspects of the invention can be used to analyze leg vasculature to detect deep leg vein thrombosis. In people who receive treatment for deep leg vein thrombosis, the rate of pulmonary embolism falls to from a high of about 50% to less than 5%. Aspects of the invention also can be used to confirm the presence of deep leg vein thrombosis in patients who have symptoms such as leg pain or discomfort. It may be important to confirm the presence of deep leg vein thrombosis before administering an anticoagulant, because the treatment can cause adverse long-term complications.

Current techniques such as ventilation-perfusion scintigraphy, leg vein ultrasound, or pulmonary angiography are often not sufficient to establish a definitive diagnosis of pulmonary embolism or deep vein thrombosis. It should be apparent that aspects of the present invention may be used alone or in conjunction with current techniques to help detect and diagnose these conditions.

Aspects of the invention also may be useful to detect the full scope of blood vessel blockage in a subject's lung vasculature. Current techniques may detect certain blockages in large to medium sized pulmonary arteries (e.g., main, lobar and segmental). However, current techniques are of limited use for detecting blockages in sub-segmental and smaller blood vessels. Aspects of the invention may be used to detect patterns (e.g., individual structural features or distributions) indicative of blockages in these smaller blood vessels. This information can be used to optimize a subject's treatment.

Detection of lesions and/or disease locations: Lesions and/or disease locations may be detected by scanning an organ in full 3D and using disease specific vascular patterns as a way to detect the location and/or boundary of diseased tissue. By placing a 3D box around a suspicious area (e.g., one that was radiologically detected) and a disease specific vascular pattern may be used to detect the boundary of the diseased tissue.

Detection or identification of patients most likely to respond to a given therapy:

Patients that are most likely to respond to a given therapy may be identified using a combination of moderately vascular diseased tissue along with the beginning of necrotic region(s) as a way to predict patients likely to respond to therapy (e.g., an anti-angiogenic therapy or an anti-cancer therapy). In addition, an increase in volume of a necrotic region of a patient identified above may be used as confirmation of a positive response to therapy.

Cancer/Angiogenesis:

Aspects of the invention may be used for tissue discrimination (e.g., for discriminating between normal and tumor tissue). In some embodiments, the presence of vessels alone may not be sufficiently informative and tissue and/or tumor-specific vascular patterns may be identified and used for analysis according to methods of the invention. In some embodiments, malignant and non-malignant soft tissue may be distinguished from each other (e.g., a benign cyst versus a tumor in a subject's breast; a benign versus a malignant lymph node in mediastinum). Parameters that may be used for discrimination may include, but are not limited to, one or more of the following: vascular diameter, vascular density (volume vessels/volume tumor), distribution curve of vascular diameters, inter-vessel distance, variability in vascular diameter, tortuosity, curvature, branching density, etc.

Aspects of the invention also may be used for therapeutic monitoring. This may involve quantification of one or more vasculature parameters. However, since the comparator is the same tumor or tissue prior to and after therapy, this monitoring may be accomplished without using specific patterns for identification of different tissues and/or tumors. In one embodiment, changes in vasculature pre- and post-therapy may be quantified (e.g., for previously identified, large (>1 cm) tumors in humans and large (>0.5 cm) tumors in mice). Parameters that may be used for therapeutic monitoring may include, but are not limited to, one or more of the following: vascular diameter, distribution of diameters, vascular density, inter-vessel distance, branching density, variability in vascular diameter (e.g., looking for “normalization”), tortuosity, curvature, etc. A therapeutic treatment may be evaluated on the basis of normalization (e.g., the score or quantitative measurement of the parameter returns towards a normal as opposed to a diseased level) of one or more of these parameters.

### Example 2

### Xenotopic Tumor Models

A tumor model can be generated by inoculating human non-small cell lung tumor cell line (A549 from ATCC, Inc.) subcutaneously in immunodeficient mice (SCID). SCID male mice (6-8 weeks old from Charles River Inc.) are inoculated subcutaneously in the lower back with a suspension of 1×10⁶ human lung tumor cells (A549) in 0.2 ml of PBS. All mice are fed normal chow diet throughout the duration of the experiment. All mice weights are measured throughout the experiment. Tumor size is measured with calipers twice-a-week and tumor volume is calculated using the formula Length²×Width×0.52. All mice are randomized into two treatment groups (approximately 10 mice per group) when the median tumor volume reaches approximately 500 mm³. The treatment groups can be treated according to the following schedule using intraperitoneal (i.p.) administration of either a control composition or an anti-angiogenic compound. For example, different levels of an anti-angiogenic compound can be used and the results compared to a control group that is not treated with an anti-angiogenic compound (e.g., Avastin® available from Genentech, South San Francisco, Calif.). For example:

Group 1: Control group—treated with saline/PBS twice a week.

Group 2: High Avastin®—treated with Avastin® at 5 mg/kg/i.p. twice a week.

Group 3: Low Avastin®—treated with Avastin® at 0.5 mg/kg/i.p. twice a week.

Experiments are terminated 1.5 weeks after initial treatment.

At the end-point, all mice are anesthetized and systemically perfused with a casting agent.

### Example 3

### Perfusion with Casting Agent

Perfusion with a casting agent, Mercox (available from Ladd Research, Williston, Vt.) can be performed as follows. An initial anticoagulation step for each animal is performed using an i.v. injection of heparin (10,000 U/ml, 0.3 cc/mouse). After 30 minutes, the animals are anesthetized. Each animal's heart is cannulated and the animal perfused with warm physiological saline at physiological pressure (with an open vein draining the organ or with an open vena cava). Perfusion is continued until the organ or animal is clear of blood. Mercox monomer is filtered through a 0.5 μm filter and a casting resin is prepared by mixing 8 ml Mercox, 2 ml methylmethacrylate, and 0.3 ml catalyst. The resin is infused through the same cannula until the onset of polymerization (the resin changes color to brown and emits heat, ˜10 min). The organ or animal is carefully immersed in a 60° C. water bath for 2 hours (or overnight in a sealed container). The tissue is removed by incubating in alternating rinses of 5% KOH and distilled water (for example in a 60° C. water bath sealed) followed by thorough rinsing in distilled water. The cast is cleaned in 5% formic acid for 15 minutes and rinsed thoroughly in distilled water and frozen in distilled water. The resulting block of ice is lyophilized (care should be taken not to melt the ice, the ice should melt as it lyophilizes). The resulting cast can be analyzed to identify one or more structural characteristics of interest.

### Example 4

### Xenotopic Tumor Models Response to Anti-Angiogenic Therapy

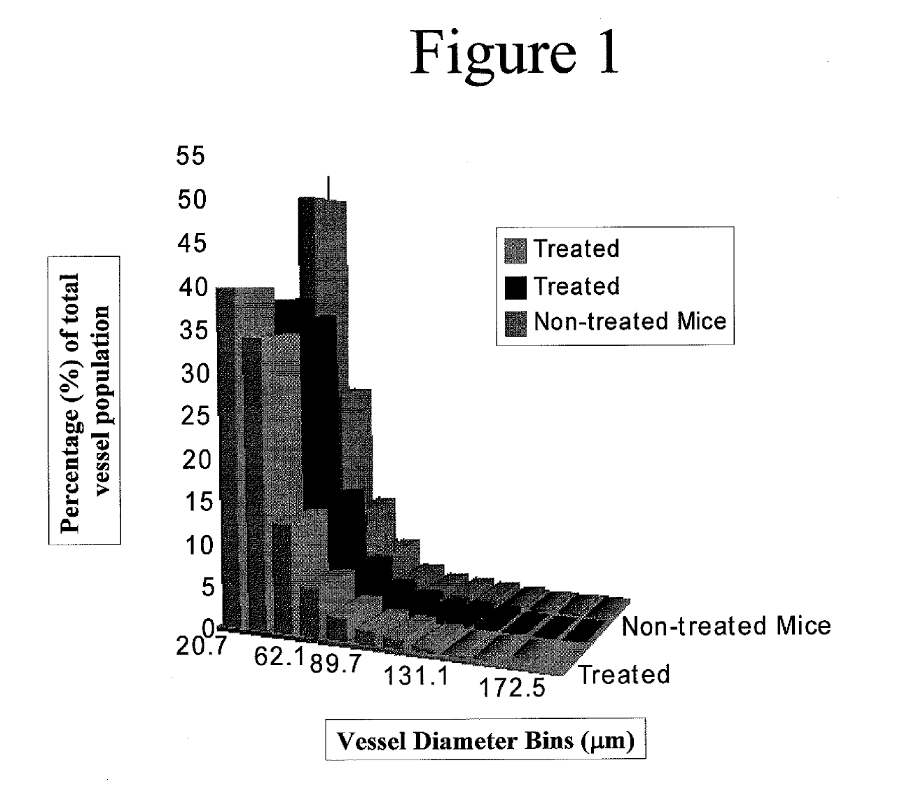

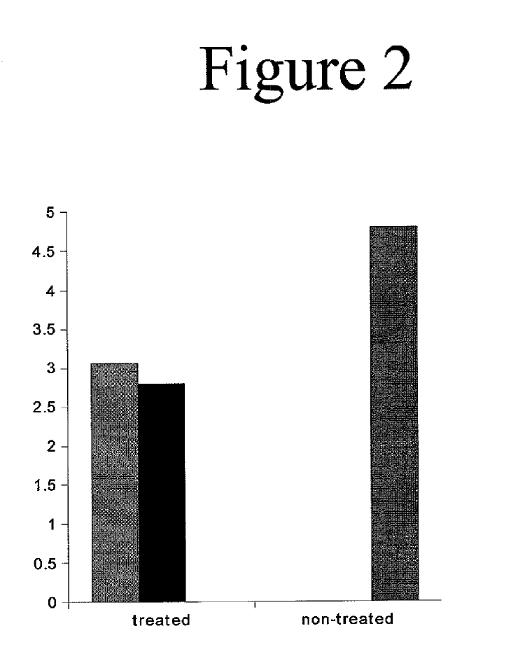

Xenotopic mouse models obtained as described in Example 2 were treated with either a control solution of saline/PBS or an anti-angiogenic preparation of Avastin® at 0.5 mg/kg/i.p. as described above. At the end-point, vascular casts were prepared as described in Example 3 above and analyzed for two treated mice (both treated with Avastin® at 0.5 mg/kg/i.p.) and one control mouse. The resulting vascular casts were scanned using a micro CT-scanner and the results of the structural analysis are shown in FIGS. 1-3. The analysis was performed by determining the number of blood vessels within bins of different diameter ranges for the xenotopic tumor in the treated and control animals. The bins were each 13.8 μm wide and the smallest bin included blood vessels having a diameter of between 20.7 μm and 34.5 μm. Mean tumor volumes did not differ significantly between the groups at the end of the experiment. However differences in blood vessel diameter distributions were detected as shown in FIGS. 1-3. FIG. 1 shows the resulting vessel population distribution. Treated tumors had 20% less small diameter sized vessels than untreated tumors, and treated tumors had a higher percentage of middle diameter sized vessels than untreated tumors. The blood vessel population distributions were consistent for both treated animals. FIG. 2 shows the vessel population ratio between small (approximately 21-35 μm) and middle (approximately 35-49 μm) size vessels in the tumors of the control and treated animals. The ratio decreased after inhibitor treatment with Avastin®, and this ratio was consistent within the treated group.

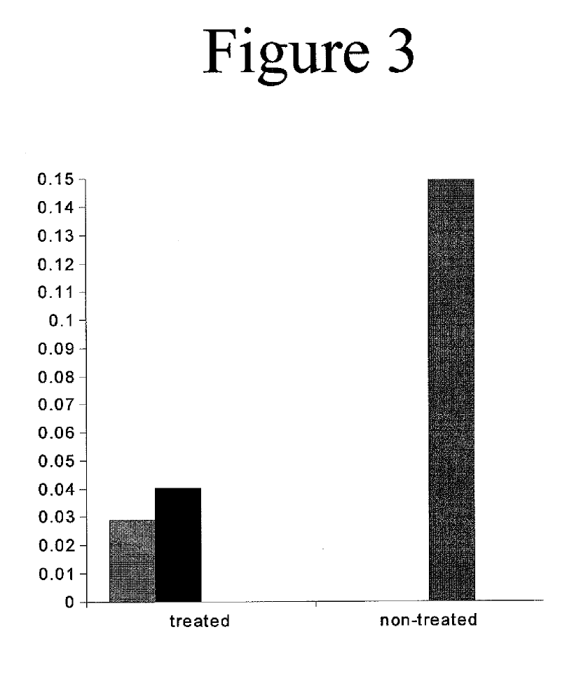

FIG. 3 shows the vessel population ratio between large (approximately 147-161 μm) and middle (approximately 33-77 μm) size vessels. The ratio decreased after treatment with Avastin®, and this ratio was consistent within the treated group.

The following considerations apply to the specific examples and the entire written specification herein (including the summary, detailed description, and claims). It should be appreciated that casts, like in situ blood vessels, are three-dimensional structures. Accordingly, imaging and analytical techniques described herein provide information about three-dimensional structural characteristics. In some embodiments, techniques are used to generate three-dimensional representations of vascular casts and/or in situ blood vessels. In some embodiments, techniques are used to generate three-dimensional images of vascular casts and/or in situ blood vessels. The three-dimensional representations and/or images can be analyzed as described herein. However, it should be appreciated that aspects of the invention are not limited to three-dimensional structural characteristics. In some embodiments, certain aspects of vascular casts and/or in situ blood vessels may be represented and/or imaged in one or two dimensions and an analysis of one or two-dimensional features may be performed and used for the applications described herein. It also should be appreciated that the structural features described herein may be measured or quantified using any appropriate units, including numbers, lengths or distances, angles, percentages, etc., or any combination thereof, further including any of these units as a function of volume or area. Similarly, it should be appreciated that vascular changes over time or in response to treatment may involve an increase or a decrease of one or more of these structural features. For example, an increase in structures associated with angiogenesis may be associated with certain disease progressions. In contrast, a decrease in structures associated with angiogenesis may be associated with disease regression (e.g., in response to treatment).

It also should be appreciated that comparisons and/or analyses described herein may involve a statistical analysis using one or more standard statistical techniques to determine whether a change in a structure or pattern or other characteristic described herein (e.g., an increase or decrease over time, or in response to a therapeutic drug), or a difference or similarity between two structures or patterns or other characteristics described herein are statistically significant.

Having thus described several aspects of at least one embodiment of this invention, it is to be appreciated various alterations, modifications, and improvements will readily occur to those skilled in the art. Such alterations, modifications, and improvements are intended to be within the spirit and scope of the invention. Any suitable analytical techniques may be used for analyzing perfused tissue and organs according to the methods described herein, including for example, the analytical techniques that are described in PCT US2005/047081 and PCT US2007/026048 the disclosures of which are incorporated herein by reference in their entirety. Accordingly, the foregoing description and embodiments are by way of example only. In the event of conflict between different disclosures, the disclosure of the present application shall control.

What is claimed is:

1. A method of identifying a vascular pattern or change in vascular pattern associated with a condition, the method comprising:
perfusing an organ or tissue of an animal with a casting agent;
analyzing a vascular structure that is perfused with the casting agent; and
identifying a vascular pattern or change in vascular pattern associated with a condition of the animal.

2. The method of claim 1, wherein the animal is an animal model of a disease.

3. The method of claim 1, wherein the animal is treated with a therapeutic drug.

4. The method of claim 1, wherein the animal has been exposed to a disease-causing agent or factor.

5. The method of claim 1, wherein the casting agent is a contrast agent.

6. The method of claim 1, wherein a contrast agent is added to the casting agent.

7. The method of claim 1, wherein the casting agent comprises a modified acrylic casting material.

8. The method of claim 1, wherein the casting agent comprises a silicone material.

9. The method of claim 1, wherein the casting agent is a polymer.

10. The method of claim 1, wherein the animal is a mouse.

11. The method of claim 1, wherein the tissue or organ is perfused in vivo.

12. The method of claim 1, wherein the tissue or organ is perfused ex vivo.

13. The method of claim 11 or 12, wherein the vasculature of the perfused tissue or organ is analyzed in situ.

14. The method of claim 11 or 12, wherein the vasculature of the perfused tissue or organ is analyzed after removal from the animal.

15. The method of claim 1, wherein the analysis is performed automatically.

16. The method of claim 7, wherein the modified acrylic casting material is Mercox®.

17. The method of claim 8, wherein the silicone material is Microfil®.

18. A method of detecting a disease in a subject, the method comprising analyzing an in situ vasculature to determine whether one or more vascular patterns of a disease are present, wherein the one or more vascular patterns are blood vessel characteristics that were correlated with the disease in a vascular cast.

19. A method for evaluating the effectiveness of a disease treatment in a subject, the method comprising
identifying changes in an in situ vasculature in response to a treatment,
comparing the changes to one or more predetermined changes in vascular pattern associated with a positive response to a disease treatment, wherein the one or more predetermined changes in vascular pattern were correlated with a positive response to treatment for a disease by analyzing a vascular cast of a disease model.

20. A method for evaluating the toxicity of a disease treatment in a subject, the method comprising
identifying changes in an in situ vasculature in response to a treatment,
comparing the changes to one or more predetermined changes in vascular pattern associated with a toxic response to a disease treatment, wherein the one or more predetermined changes in vascular patterns were correlated with a toxic response to treatment for a disease by analyzing a vascular cast of a disease model.

21. The method of any one of claims 18-20, wherein the subject is human.

22. The method of any one of claims 18-20, further comprising the step of identifying the one or more predetermined structural characteristics or changes therein by analyzing a vascular cast of a disease model.

23. The method of claim 22, wherein the disease model is an animal model.

24. The method of claim 23, wherein the animal model is an ectopic or orthotopic tumor model.

25. The method of claim 1, further comprising comparing the vascular pattern or change in vascular pattern to an in situ vasculature in a subject to determine whether one or more indicia of disease, responsiveness to therapy, or other condition are present in the subject.
